(19) United States
(12) Patent Application Publication
(10) Pub. No.: US 2005/0261672 A1
(43) Pub. Date: Nov. 24, 2005

(54) Systems and methods for selective denervation of heart dysrhythmias

(76) Inventors: Mark Deem, Mountain View, CA (US); Hanson S. Gifford, Woodside, CA (US); Denise Demarais, Los Gatos, CA (US)

Correspondence Address: LUCE, FORWARD, HAMILTON & SCRIPPS LLP, 11988 EL CAMINO REAL, SUITE 200, SAN DIEGO, CA, 92130, US

(21) Appl. No.: 11/133,088

(22) Filed: May 18, 2005

Related U.S. Application Data
(60) Provisional application No. 60/572,458, filed on May 18, 2004.

(51) Int. Cl. A61B 18/14

(52) U.S. Cl. 606/41; 607/122

(57) ABSTRACT

Methods and apparatus are provided for selective denervation of conduction pathways in the heart for the treatment of dysrhythmias, including one or more ablation or electroporation catheters having electrodes for stimulating, targeting, and ablating fat pad tissue and other cardiac tissue to selectively denervate heart tissue.

### REFERENCE TO RELATED APPLICATIONS

This application claims the benefit of the filing date of U.S. provisional patent application Ser. No. 60/572,458 filed May 18, 2004, which is incorporated herein by reference in its entirety.

### FIELD OF THE INVENTION

The present invention relates to methods and apparatus for the treatment of heart dysrhythmias, and more particularly, for selective denervation of conduction pathways in the heart for the treatment of dysrhythmias.

### INCORPORATION BY REFERENCE

All publications and patents or patent applications mentioned in this specification are herein incorporated by reference to the same extent as if each individual publication, patent or patent application was specifically and individually so incorporated by reference.

### BACKGROUND OF THE INVENTION

Up until the 1980s, there was dramatic growth in the creation of new surgical methods for treating a wide variety of previously untreated heart conditions. Over the past twenty years there has been a clear trend towards the invention of devices and methods that enable less invasive treatment, moving from invasive surgery to less-invasive surgery and interventional therapies. Ultimately, it is desirable to move to totally non-invasive therapies.

The history of treatment of atrial fibrillation began when Dr. James Cox invented a new open-heart surgical procedure that interrupted depolarization waves using surgical incisions in the wall of the left atrium. A number of devices have been developed to allow surgeons to make such lesions during surgery on a beating heart, without making incisions in the walls of the atrium. More recently, interventional electrophysiologists have worked with companies to develop catheter-based systems to create similar lesions.

Current ablation strategies for the treatment of atrial fibrillation involve complex ablation patterns that require extensive electroanatomical mapping, a large number of discrete ablations, and a procedure that can take upwards of eight hours to complete. Although a multitude of ablation patterns have been described, the majority of them are aimed at replicating the MAZE procedure, developed by Dr. James Cox.

Research has been aimed at reducing the number of ablations required to successfully treat atrial fibrillation. Haissaguerre, Pappone, and others have described segmental or fully circumferential pulmonary vein isolation with success rates of 50-70%. These approaches often include additional linear lesions at the mitral isthmus and/or the left atrial roof, which improved initial results to a success rate of 65-85%.

Pappone has also described selective vagal denervation as an adjunct to circumferential pulmonary vein isolation. The identification and ablation of sites that triggered vagal reflexes in the left atrium resulted in complete vagal denervation of the pulmonary veins, contributing to improved outcomes and less recurrent atrial fibrillation. Vagal reflexes were defined as sinus bradycardia (<40 bpm), asystole, AV block, or hypotension and were identified by applying radio-frequency (RF) energy. Once the reflex was evoked, RF energy was then used to ablate the site, eliminating the vagal response. Although this approach continues to utilize extensive lesion sets, it suggests the opportunity for the development of reduced ablation patterns.

Recently, others have described targeting and ablation strategies for treating atrial fibrillation and ventricular tachycardia in which both sympathetic and parasympathetic conduction pathways are eliminated. The identification of vagal reflexes is achieved by stimulating with RF energy to solicit prolonged RR intervals, asystole, or induce atrial fibrillation. These target locations are then ablated from the left atrium, requiring a significantly reduced number of total ablation sites. Although promising, this strategy continues to require a relatively invasive procedure, extensive catheter manipulation, and ablation of the left atrial wall.

An alternative approach for treating atrial fibrillation involves identification and ablation of parasympathetic (vagal) pathways to the atria, thus imparting selective parasympathetic denervation without disruption of sympathetic control. Since the mid 1980s, research has led to the identification of various “fat pads”, which contain autonomic ganglia that innervate the atria and control atrio-ventricular and sino-atrial nodal function. In patients with atrial fibrillation, these ganglia are over-active. Elimination of these fat pads in canines selectively denervated the atria, reducing the autonomic burden on the heart.

It is hypothesized that a similar ablation strategy may cure atrial fibrillation in humans. Some have suggested that part of the success of the targeted ablation technique may derive from serendipitous ablation of fat pad tissue, and that inconsistency of results may be related to incomplete fat pad ablation that results from present procedures and technologies. Unfortunately, this technique currently requires a relatively invasive procedure in which the pericardium and posterior aspect of the heart must be accessed.

In view of the aforementioned limitations, it would be desirable to provide methods and apparatus for treating ventricular tachycardia and a variety of other cardiac dyssynchronies which are minimally or non-invasive, more safe and effective, consist of a limited lesion set, and offer a shorter treatment times.

It would also be desirable to provide methods and apparatus for treating atrial fibrillation and other conduction defects by modifying intrinsic and/or extrinsic nerves of the heart.

It would further be desirable to provide methods and apparatus for treating atrial fibrillation and other conduction defects by modifying conduction pathways in the heart, thus altering the interaction between the intrinsic and extrinsic nervous systems of the heart.

It would also be desirable to provide selective ablation of sympathetic and/or parasympathetic pathways in the heart in a non-invasive or minimally invasive manner.

It would additionally be desirable to provide methods and apparatus for treating atrial fibrillation and other conduction defects by stimulating, targeting and ablating from a single or multiple locations adjacent to target areas.

It would also be desirable to provide methods and apparatus for treating atrial fibrillation and other conduction defects by stimulating, targeting and ablating from single or multiple locations at a distance from target areas, in a non-invasive procedure.

It would also be desirable to provide methods and apparatus for treating atrial fibrillation and other conduction defects by utilizing energy to disrupt tissue at the cellular level via permeabilization of the cell membrane to effect the intrinsic and/or extrinsic nerves of the heart. Depending on the amplitude and duration of the applied field, such electroporation may be reversible or irreversible, as desired. Reversible electroporation may be used in conjunction with a nerve blocking agent, chemical or other therapeutic agent to disrupt the nerves and/or tissue.

### SUMMARY OF THE INVENTION

In view of the foregoing, it is an object of the present invention to provide methods and apparatus for treating, ventricular tachycardia, and a variety of other cardiac dyssynchronies which are minimally or non-invasive, more safe and effective, consist of a limited lesion set, and offer a shorter treatment times.

Another object of the present invention is to provide methods and apparatus for treating atrial fibrillation and other conduction defects by modifying intrinsic and/or extrinsic nerves of the heart.

A further object of the present invention is to provide methods and apparatus for treating atrial fibrillation and other conduction defects by modifying conduction pathways in the heart, thus altering the interaction between the intrinsic and/or extrinsic nervous systems of the heart.

An additional object of the present invention is to provide selective ablation of sympathetic and/or parasympathetic pathways in the heart in a non-invasive or minimally invasive manner.

Yet another object of the present invention is to provide methods and apparatus for treating atrial fibrillation and other conduction defects by stimulating, targeting and ablating from a single or multiple locations adjacent to target areas.

A further object of the present invention is to provide methods and apparatus for treating atrial fibrillation and other conduction defects by stimulating, targeting and ablating from single or multiple locations at a distance from target areas, in a minimally or non-invasive procedure.

A yet further object of this invention is to provide methods and apparatus for treating atrial fibrillation and other conduction defects by utilizing an electric field generated by a pulse or pulses of a designated duration and amplitude to disrupt tissue at the cellular level via permeabilization of the cell. The use of ultrashort electric field pulses causes irreversible cell damage by creating pores in the cell membrane or intracellular electromanipulation, thereby leading to apoptosis of the targeted cell. Such cellular damage may be used to affect the intrinsic and/or extrinsic nerves of the heart.

Another object of the present invention is to provide methods and apparatus for treating atrial fibrillation and other conduction defects by utilizing an electric field to disrupt tissue at the cellular level via permeabilization of the cell causing reversible electroporation of the cellular membrane. Such reversible electroporation is applied in conjunction with a therapeutic agent such as a nerve blocking agent.

Selective denervation of sympathetic and/or parasympathetic conduction pathways in the heart may be useful in developing treatment methodologies for curing many types of cardiac dysrhythmias. Denervation of sympathetic and/or parasympathetic conduction pathways may provide a means to reduce sympathovagal tone, thus altering the autonomic burden in the heart. For example, it is believed that the sympathetic pathways provide the trigger for the induction of atrial fibrillation, and that the parasympathetic pathways provide the substrate that facilitates ongoing fibrillation. By denervating these pathways, dysrhythmias may be cured.

### BRIEF DESCRIPTION OF THE DRAWINGS

Further features of the invention, its nature and various advantages will be more apparent from the accompanying drawings and the following detailed description of the preferred embodiments, in which:

FIG. 1 is a schematic view illustrating the fat pads of a human heart;

FIG. 2 is a table demonstrating exemplary combination therapies for stimulating, targeting, and ablating fat pads;

FIG. 3 is a schematic view illustrating a combination therapy for selective denervation of the SVC-Ao fat pad in accordance with the principles of the present invention;

FIG. 4 is a schematic view illustrating a combination therapy for selective denervation of the LA fat pad in accordance with the principles of the present invention;

FIG. 5 is a table demonstrating exemplary remote location therapies for stimulating, targeting, and ablating fat pads;

FIG. 6 is a schematic view illustrating a remote location therapy for selective denervation of the CS fat pad from the esophagus in accordance with the principles of the present invention;

FIG. 7 is a schematic view illustrating a remote location therapy for selective denervation of the SVC-Ao fat pad from the superior vena cava in accordance with the principles of the present invention;

FIG. 8 depicts a cardiac electroporation catheter and pulse generator in accordance with the principles of the present invention; and

FIGS. 9A and 9B are schematic views depicting the placement and activation of an electroporation treatment catheter to selectively denervate cardiac tissue in various target regions within the heart.

### DETAILED DESCRIPTION OF THE INVENTION

The present invention is directed to methods and apparatus for stimulating, targeting, and creating lesions in the walls of the heart in order to selectively denervate nerve bundles which make up conduction pathways responsible for atrial fibrillation and other cardiac dysrhythmias. Target tissue may be ablated from one or more locations either adjacent to or at a distance from target tissue. The target tissue may include conduction pathways associated with either or both the intrinsic and extrinsic nerves of the heart. Dysrhythmias that may be treated using this technology include, but not limited to, atrial flutter, atrial fibrillation, atrial tachycardia, bradycardia, ventricular tachycardia, atrial/ventricular dyssynchrony, and ventricular/ventricular dyssynchrony.

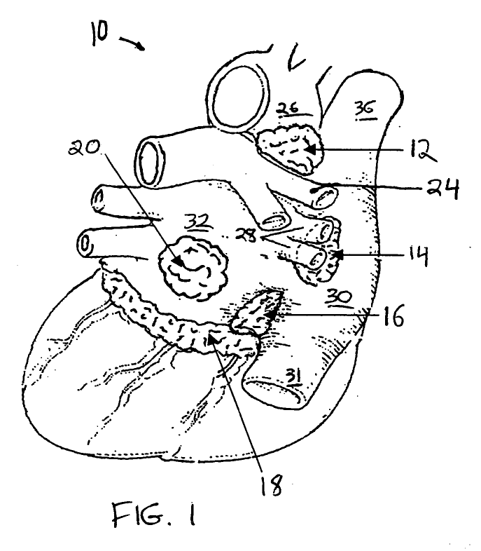

Referring to FIG. 1, the posterior aspect of a human heart 10 is shown with the approximate location of the various fat pads, including superior vena cava and aortic root (SVC-Ao) fat pad 12, right pulmonary vein-atrial (RPV) fat pad 14, inferior vena cava-left atrial (IVC-ILA) fat pad 16, coronary sinus (CS) fat pad 18, and left atrium (LA) fat pad 20. SVC-Ao fat pad 12 is located just anterior to right pulmonary artery 24 at the root of aorta 26. RPV fat pad 14 overlies and partially surrounds right pulmonary veins 28 near the entrance to right atrium 30. IVC-ILA fat pad 16 lies at the junction of inferior vena cava 31, the inferior left atrium, and the ostium of the coronary sinus. CS fat pad 18 traverses along the length of the coronary sinus, while LA fat pad 20 covers a large portion of the dorsal surface of the left atrium 32. Selective transmural ablation of one or more of these fat pads denervates the atria of the heart, thereby providing a cure for atrial fibrillation.

In accordance with the principles of the present invention, selective denervation of heart tissue for the treatment of atrial fibrillation and other conduction defects is achieved by stimulation, targeting, and ablating fat pad tissue from one or more adjacent structures including the vasculature, heart, and esophagus. By way of example, denervation of SVC-Ao fat pad 12 may be achieved by stimulating, targeting, and ablating tissue from a single location such as right pulmonary artery 24, superior vena cava (SVC) 36, the pericardium, aorta 26, or the esophagus. Similarly, RPV fat pad 14 may be treated from right pulmonary veins 28, right atrium 30, left atrium 32, the pericardium, or the esophagus.

Denervation of IVC-ILA fat pad 16 may be achieved by stimulating, targeting, and ablating tissue from a single location such as inferior vena cava 31, right atrium 30, left atrium 32, the coronary sinus, the pericardium, or the esophagus. Likewise, LA fat pad 20 may be treated from left atrium 32, the pericardium, or the esophagus, whereas CS fat pad 18 may be treated from left atrium 32, the coronary sinus, the pericardium, or the esophagus. As would be understood to those of skill in the art, other fat pads and relevant conduction pathways that are not yet identified are intended to be within the scope of the present invention.

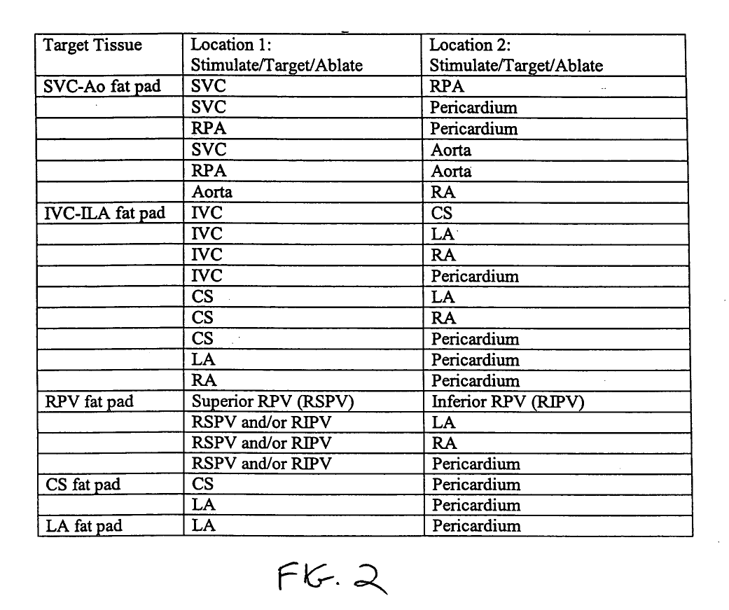

According to an aspect of the present invention, fat pads 12, 14, 16, 18, 20 are stimulated, targeted, and ablated from more than one adjacent structure to ensure complete ablation. Advantageously, ablation of target tissue using combination treatment strategies potentially requires less energy that other treatments. Referring to FIG. 2, exemplary combination treatment strategies that incorporate adjacent structures are provided. Combination locations for ablating SVC-Ao fat pad tissue include, but are not limited to: (1) the superior vena cava (SVC) and the right pulmonary artery (RPA); 2)the SVC and aorta; (3) the RPA and aorta; and (4) the aorta and right atrium (RA).

With further reference to FIG. 2, exemplary combination treatment locations for ablating IVC-ILA fat pad tissue include, but are not limited to: (1) the inferior vena cava (IVC) and the coronary sinus (CS); (2) the IVC and left atrium (LA); (3) the IVC and RA; (4)the IVC and pericardium; (5) the CS and LA; (6) the CS and RA; (7) the CS and pericardium; (8) the LA and pericardium; (9) the RA and pericardium. Similarly, exemplary combination treatment locations for ablating RPV fat pad tissue include, but are not limited to: (1) the right superior pulmonary vein (RSPV) and the right inferior pulmonary vein (RIPV) (2) the RSPV and LA; (3) the RSPV and RA; (4) the RIPV and LA; (5) the RIPV and RA; and (6) the RIPV and pericardium. Like wise, exemplary combination treatment locations for ablating CS fat pad tissue include, but are not limited to: (1) the CS and the pericardium (CS); and (2) the LA and the pericardium; whereas exemplary combination treatment locations for ablating LA fat pad tissue include, but are not limited to the LA and the pericardium. As would be appreciated by those of skill in the art, additional treatment location combinations and target tissues are possible, and are intended to be within the scope of the present invention.

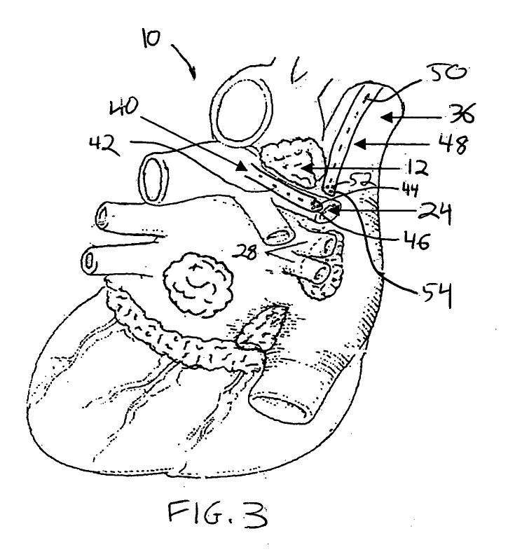

Referring to FIG. 3, a system and method for selective denervation of heart tissue by abating the SVC-Ao fat pad will now be described. In the illustrated embodiment, SVC-Ao fat pad 12 is treated from both the right pulmonary artery 24 and superior vena cava 36. An ablation system comprises ablation catheter 40 having lumen 42 and one or more electrodes 44 disposed at or near distal tip 46, and ablation catheter 48 having lumen 50 and one or more electrodes 52 disposed at distal tip 54. Electrodes 44, 52 employ energy (e.g., RF energy) for stimulating, targeting, and/or ablating target tissue. Electrodes may be powered by electrical wires running through lumens 42, 50. Stimulation, targeting and ablation of fat pads may also be accomplished using microwaves, cryothermia probes/balloons, alcohol injection, laser light, magnetic stimulation, and/or ultrasound energy.

In operation, catheter 40 is inserted percutaneously and advanced into right pulmonary artery 24, while catheter 42 inserted percutaneously and advanced into superior vena cava 36. According to some embodiments, catheter 40 further comprises an expansion element 62 (e.g., an expandable balloon or umbrella) disposed generally at or near distal tip 54. In this case, catheter 40 is inserted percutaneously and guided into right atrium, and then expansion element 58 is expanded such that blood flow directs the catheter into the right ventricle and subsequently into right pulmonary artery 24. Once the catheters have been appropriately positioned, stimulation using either or both catheters is used to elicit a vagal reflex. Stimulation may be achieved through electrical, magnetic, or other energy application. By observing the vagal reflex, a target ablation location is determined, and then ablation is employed to eliminate the vagal reflex, thereby selectively denervating the conduction pathways. Alternatively, visualization and targeting of the fat pad via ultrasound or other suitable means may be achieved through visualization apparatus built into either or both catheters 40 and/or 42.

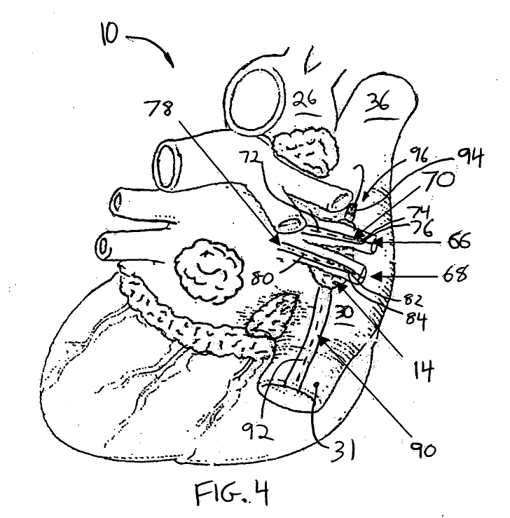

Referring to FIG. 4, a system and method for selective denervation of heart tissue by abating the RPV fat pad will now be described. In the illustrated embodiment, RPV fat pad 14 is treated from both the right atrium 30 and right superior pulmonary vein 66 and/or right inferior pulmonary vein 68. An ablation system comprises catheter 70 having lumen 72 and one or more electrodes 74 disposed at or near distal tip 76, catheter 78 having lumen 80 and one or more electrodes 82 disposed at or near distal tip 84. According to some embodiments, an additional ablation catheter 90 having lumen 92 and one or more electrodes 94 disposed at or near distal tip 96. Electrodes 74, 82, 94 employ energy (e.g., RF energy) for stimulating, targeting, and/or ablating target tissue. Electrodes may be powered by electrical wires running through lumens 72, 80, 92.

In operation, catheter 70 is inserted percutaneously and advanced into either the right superior pulmonary vein 66 or the right inferior pulmonary vein 68. Catheter 78 optionally may then be inserted percutaneously and advanced into the other pulmonary vein. Similarly, catheter 90 is inserted percutaneously and advanced into right atrium 30, on the opposing side of RPV fat pad 14. Once the catheters have been appropriately positioned, stimulation using either or both catheters is used to elicit a vagal reflex. By observing the vagal reflex, a target ablation location is determined, and then ablation using both catheters is employed to eliminate the vagal reflex, thereby selectively denervating the conduction pathways. Alternatively, visualization and targeting of the fat pad via ultrasound or other suitable means may be achieved through visualization apparatus built into either or both catheters 78 and/or 90.

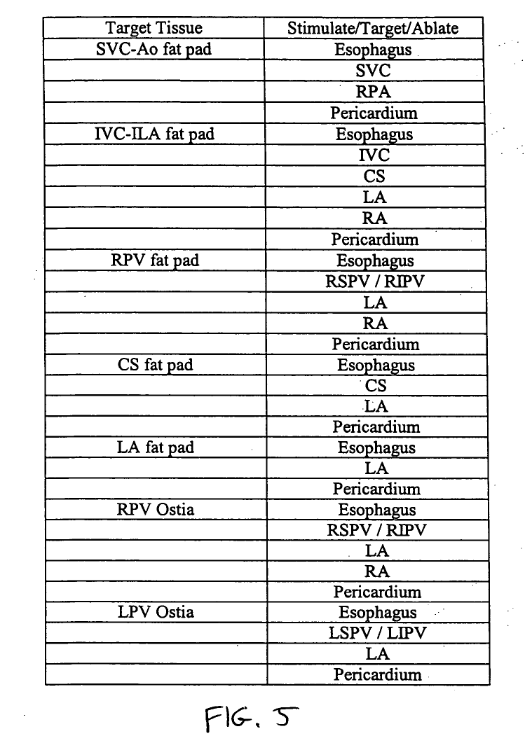

Selective denervation of conduction pathways for the treatment of cardiac dysrhythmias may also be achieved by stimulating, targeting, and ablating fat pads and other conduction pathways from one or more remote locations. Targeting may include visualization of target structures by ultrasound or other appropriate visualization technology, electrical identification, or other targeting means. Referring to FIG. 5, exemplary remote location treatment strategies for ablating fat pads for the treatment of cardiac dysrhythmias are provided. Remote locations for ablating SVC-Ao fat pad tissue include, but are not limited to: (1) the esophagus; (2) the RPA; (3) the SVC and (4) the aorta. Similarly, exemplary remote location treatment strategies for ablating IVC-ILA fat pad tissue include, but are not limited to: (1) the esophagus; (2) the IVC; (3) the CS; (4) the LA; (5) the RA; and (6) the pericardium. Likewise, exemplary remote locations for ablating RPV fat pad tissue include, but are not limited to: (1) the esophagus; (2) the RSPV; (3) the RIPV; (4) the LA; (5) RA; and (6) the pericardium.

With further reference to FIG. 5, exemplary remote location treatment strategies for ablating LA fat pad tissue include, but are not limited to: (1) the esophagus; (2) the LA; and (3) the pericardium. Similarly, exemplary remote locations for ablating CS fat pad tissue include, but are not limited to: (1) the esophagus; (2) the CS; (3) the LA; and (4) the pericardium. Likewise, exemplary remote locations for ablating the RPV ostia include, but are not limited to: (1) the esophagus; (2) the RSPV; (3) the RIPV; (4) the LA; (5) RA; and (6) the pericardium. Moreover, exemplary remote locations for ablating the LPV ostia include, but are not limited to: (1) the esophagus; (2) the LSPV; (3) the LIPV; (4) the LA; and (5) the pericardium.

FIG. 5 provides treatment strategies in which stimulation, targeting, and/or ablation is performed using one or more devices located at a distance from the target tissue. As would be understood by those of skill in the art, additional remote locations treatment strategies are possible, and are intended to be within the scope of the present invention. For example, it has been shown that cardiac imaging (transesophageal electrocardiography (TEE)), pacing and defibrillation can be accomplished via a transesophageal approach. Further, methods and apparatus have been disclosed for ultrasound imaging and high-frequency ultrasound (HIFU) ablation of target tissue via a transesophageal approach. Such methods and apparatus are described in U.S. Provisional Application Ser. No. 60/477,532 (filed Jun. 10, 2003), the contents of which are incorporated herein by reference. It is, therefore, possible to remotely target and ablate tissue from other remote locations, such as the great vessels, pulmonary veins/arteries, coronary sinus, atria, and the pericardium.

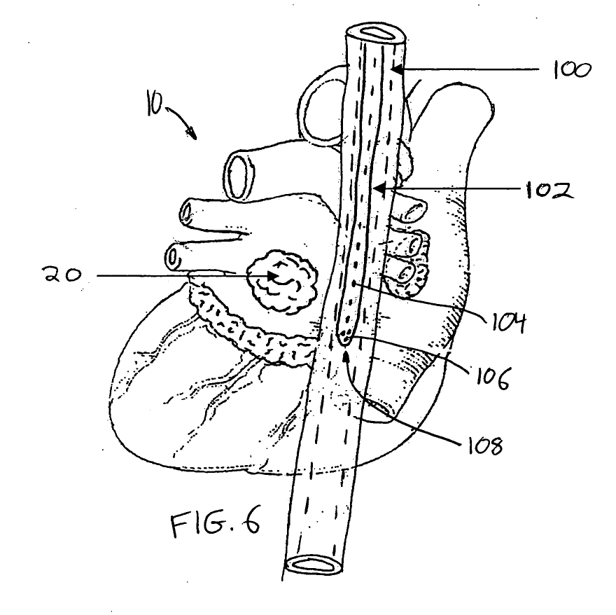

Referring to FIG. 6, a system and method for selective denervation of heart tissue by abating the LA fat pad will now be described. In the illustrated embodiment, LA fat pad 20 is treated from esophagus 100 using an ablation system comprising one or more catheters 102 having lumen 104 and one or more electrodes 106 disposed at or near distal tip 108. Electrodes 106 use energy to stimulate, target, and/or ablate target tissue. Electrodes are powered via electrical wires running through lumen 104. In operation, catheter 102 is positioned within esophagus 100 above the level of the coronary sinus and used to stimulate, target, and/or ablate LA fat pad 20.

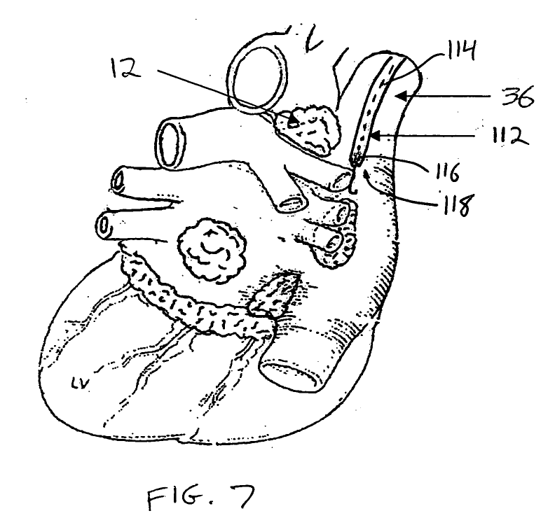

Referring to FIG. 7, a system and method for selective denervation of heart tissue by abating the SVC-Ao fat pad will now be described. In the illustrated embodiment, SVC-Ao fat pad 12 is treated from superior vena cava 36 using an ablation system comprising one or more catheters 112 having lumen 114 and one or more electrodes 116 disposed at distal tip 118. Electrodes 116 use energy to stimulate, target, and/or ablate target tissue. Electrodes are powered via electrical wires running through lumen 114. In operation, catheter 112 is positioned within superior vena cava 36 near the junction with right atrium 30 and used to stimulate, target, and/or ablate LA fat pad 20.

It may be desirable to position an adjunctive device in a second location to aid in the stimulation, targeting, and/or ablation of target tissue. For example, with further reference to FIG. 7, it may be advantageous to position a second catheter in the right pulmonary artery to aid in targeting the SVC-Ao fat pad. It may also be desirable to place a catheter in the superior vena cava to stimulate SVC-Ao fat pad 12, RPV fat pad 14, and IVC-ILA fat pad 16, while a second catheter is positioned within esophagus 100 to provide targeting and/or ablation of all three of these fat pads.

Research has shown that a majority of the autonomic nerves pass through the SVC-Ao fat pad, which then go on to innervate both the RPV and IVC-ILA fat pads. Thus, by stimulating SVC-Ao fat pad 12, both RPV fat pad 14 and IVC-ILA fat pad 16, may be stimulated and targeted. As would be understood to those of skill in the art, many such fat pad combinations exist, and are intended to be within the scope of the present invention.

In addition, to achieve the goals of the present invention, it may be desirable to employ methods and apparatus for achieving cardiac nerve modulation and/or denervation utilizing pulsed electric fields and/or electroporation applied directly to the targeted region or in proximity to the targeted region to produce the desired denervation or nerve disruption. For purposes of this disclosure, the term “electroporation” encompasses the use of pulsed electric fields (PEFs), nanosecond pulsed electric fields (nsPEFs), ionophoreseis, electrophoresis, electropermeabilization, sonoporation and/or combinations thereof. Further, the term “ablation” in this specification may be read to encompass the mechanism of electroporation leading to denervation whether it be, permanent or temporary, reversible or irreversible, with or without the use of adjuctive agents, without necessitating the presence of a thermal effect.

Reversible electroporation, first observed in the early 1970's, has been used extensively in medicine and biology to transfer chemicals, drugs, genes and other molecules into targeted cells for a variety of purposes such as electrochemotherapy, gene transfer, transdermal drug delivery, vaccines, and the like. Irreversible electroporation, although avoided for the most part historically when using electroporation techniques, has more recently been used for cell separation in such applications as debacterilization of water and food, stem cell enrichment and cancer cell purging (U.S. Pat. No. 6,043,066 to Mangano), directed ablation of neoplastic prostate tissues (US2003/0060856 to Chornenky), treatment of restenosis in body vessels (US2001/0044596 to Jaafar), selective irreversible electroporation of fat cells (US 2004/0019371 to Jaafar) and ablation of tumors (Davalos, et al Tissue Ablation with Irreversible Electroporation, Annals of Biomedical Engineering 33:2, pp. 223-231 (February 2005), the entire contents of each are expressly incorporated herein by reference.

Further, energy fields applied in ultrashort pulses, or nanosecond pulsed electric fields (nsPEFs) have application to the present invention. Such technology utilizes ultrashort pulse lengths to target subcellular structures without permanently disrupting the outer membrane. An example of this technology is described by Schoenbach et al. in Intracellular Effect of Ultrashort Electrical Pulses in J. Bioelectromagnetics 22:440-448 (2001), and further described in U.S. Pat. No. 6,326,177, the contents of which is expressly herein incorporated by reference. The short pulses target the intracellular apparatus, and although the cell membrane may exhibit an electroporative effect, such effect is reversible and does not lead to permanent membrane disruption. Following application of nanosecond pulses apoptosis is induced in the intracellular contents, affecting the cell's viability (for example the ability to reproduce).

In general, electroporation may be achieved utilizing a device adapted to activate an electrode set or series of electrodes to produce an electric field. Such a field may be generated using either a bipolar or monopolar electrode configuration. When applied to cells, depending on the duration and strength of the applied pulses, this field operates to increase the permeabilization of the cell membrane and either: 1) reversibly open the cell membrane for a short period of time by causing pores to form in the cell lipid bilayer allowing entry of various therapeutic elements or molecules, after which, when energy application ceases, the pores spontaneously close without killing the cell; 2) irreversibly open or porate the cell membrane causing cell instability resulting in cell death utilizing higher intensity (longer or higher energy) pulses; or 3) applying energy in nanosecond pulses resulting in disruption of the intracellular matrix leading to apoptosis and cell death, without causing irreversible poration of the cellular membrane. As characterized by Weaver, Electroporation: A General Phenomenon for Manipulating Cells and Tissues Journal of Cellular Biochemistry, 51:426-435 (1993), short(1-100 μs) and longer (1-10 ms) pulses have induced electroporation in a variety of cell types. In a single cell model, most cells will exhibit electroporation in the range of 1-1.5V applied across the cell (membrane potential). For applications of electroporation to cell volumes, ranges of 10 V/cm to 10,000 V/cm and pulse durations ranging from 1 nanosecond to 0.1 seconds may be applied.

Certain factors effect how a delivered electric field will effect a targeted cell, including cell size, cell shape, cell orientation with respect to the applied electric field, cell temperature, distance between cells (cell-cell separation), cell type, tissue heterogeneity, properties of the cellular membrane and the like. Larger cells may be more vulnerable to injury. For example, skeletal muscle cells have been shown to be more susceptible to electrical injury than nearby connective tissue cells (Gaylor et al. Tissue Injury in Electrical Trauma, J. Theor. Biol. (1988) 133, 223-237). In addition, how cells are oriented within the applied field can make them more susceptible to injury, for example, when the major axis of nonspherical cells is oriented along the electric field, it is more susceptible to rupture (Lee et al, Electrical Injury Mechanisms: Electrical Breakdown of Cell Membranes, Plastic and Reconstructive Surgery, November 1987, 672-679.)

Various waveforms or shapes of pulses may be applied to achieve electroporation, including sinusoidal AC pulses, DC pulses, square wave pulses, exponentially decaying waveforms or other pulse shapes such as combined AC/DC upulses, or DC shifted RF signals such as those described by Chang in Cell Poration and Cell Fusion using and Oscillating Electric Field, Biophysical Journal October 1989, Volume 56 pgs 641-652, depending on the pulse generator used or the effect desired. The parameters of applied energy may be varied, including all or some of the following: waveform shape, amplitude, pulse duration, interval between pulses, number of pulses, combination of waveforms and the like.

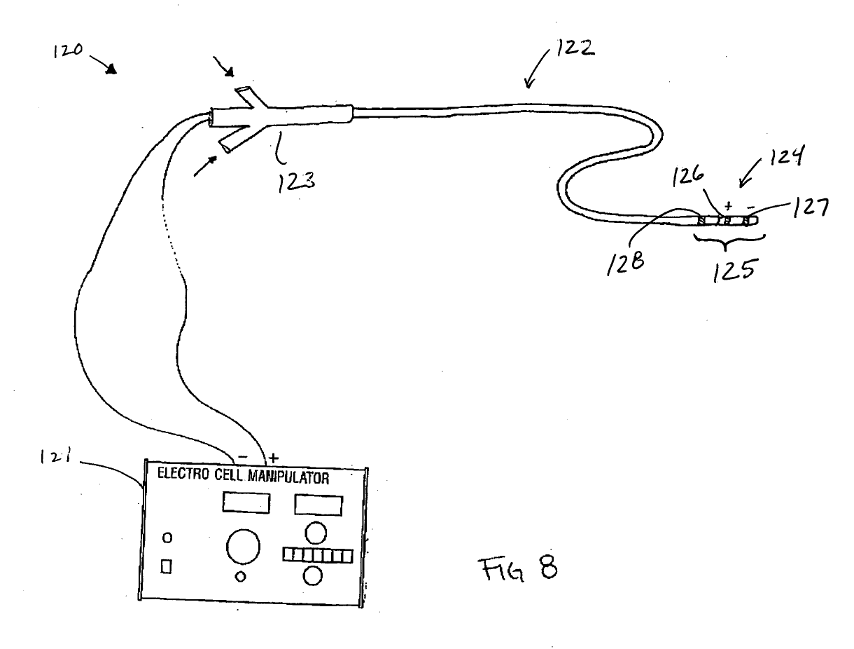

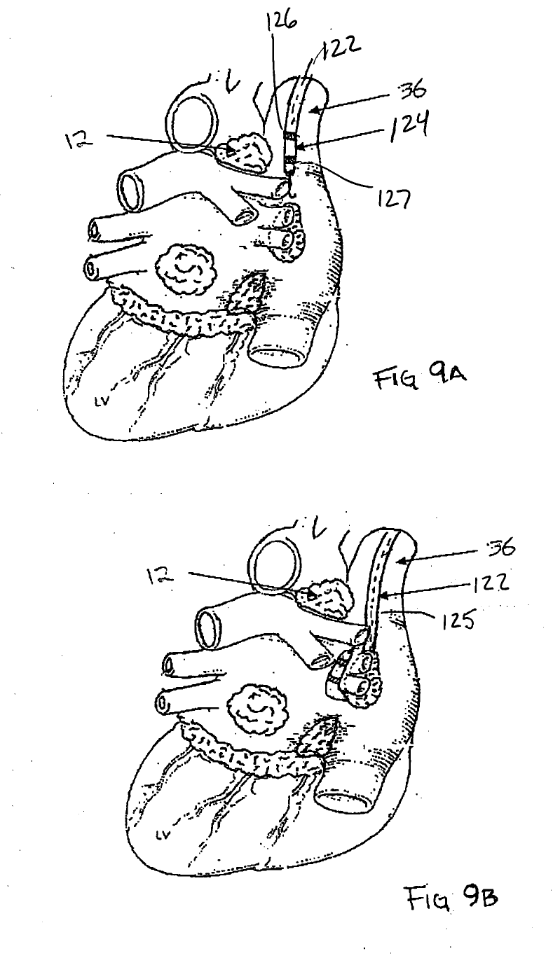

Referring to FIGS. 8 and 9A-9B, a system and method utilizing an electroporation catheter for selective denervation/disruption of heart tissue is described. Further descriptions of vascular electroporation catheters are described in U.S. patent application 2001/0044596 filed May 4, 2001 and US2002/0040204 filed Dec. 15, 2000, the full disclosures of which are expressly incorporated herein by reference in their entireties.

In FIG. 8, electroporation catheter system 120 comprises pulse generator 121 such as a model PA-2000S or PA-4000S available from Cytopulse Sciences, Inc. Columbia, Md. or the Gene Pulser Xcell, Bio-Rad, Inc. Pulse generator 121 is electrically coupled to intravascular catheter 122 having proximal end 123 and distal end 124. Catheter 122 is configured for minimally invasive insertion into a desired region of the heart as described herein below, and includes electroporation element 125 disposed at distal end 126.

Electroporation element 125 includes first electrode 126 and second electrode 127 operatively connected to pulse generator 121 for delivering a desired number, duration, amplitude and frequency of pulses to targeted cardiac tissue. These parameters may be modified either by the system or the user, depending on the location of the catheter within the heart, e.g., with regard to intervening tissues or structures, and whether a reversible or irreversible cell poration is desired.

For example energy in the range of 10 to 10,000 V/cm for a duration of 10 μs may be used to achieve reversible electroporation, and in the range of approximately 100 to 1,000,000 V/cm to achieve irreversible electroporation. An additional mapping electrode or electrodes 128, may be located on the catheter shaft near distal end 124.

In operation, the effects of electroporation on heart tissue may be selected depending on the type of tissue targeted. For example, fat cells located within the fat pad described above may be more susceptible to damage and thus a lower voltage may be applied when directing energy to these cells so as not to affect surrounding muscle tissue. Similarly, nerve cells targeted in the region of the pulmonary veins or within heart muscle may be preferentially affected due to size, sparing smaller or cross-oriented muscle tissue.

Referring now to FIGS. 9A and 9B, methods of cardiac ablation using the electroporation catheter of FIG. 8 to ablate a patient's SVC-Ao fat pad is described. Electroporation catheter 122 illustratively is introduced via superior vena cava 36 to the location of the target tissue. In use, electroporation catheter 130 need not be placed only via the SVC 12, but may be placed in a manner similar to those herein described in FIGS. 4 and 5. Once positioned adjacent the cardiac tissue to be treated, pulse generator 121 (see FIG. 8) may be activated, causing an electric field to be generated in the target area using electrodes 126 and 127.

It is further within the scope of the present invention to use the electroporation catheter of the present invention to perform reversible cell permeabilization utilizing a therapeutic agent, or irreversible cell permeabilization to induce cell death, in regions of the heart where traditional ablative techniques are applied, for example in the region of the pulmonary veins or other regions such as linear lesions at the mitral isthmus and/or left atrial roof, that replicate the MAZE procedure as previously described.

In FIG. 9B, electroporation catheter 122 is introduced via the superior vena cava 36 to the location of the target area, specifically the pulmonary veins. Catheter 122 is then manipulated to direct the electroporation element 125 to surround the pulmonary veins prior to activating the electric field. Pulse generator 121 may be synchronized with the heart beat to maximize delivery of the energy at the desired interval of the cardiac cycle by gating the treatment to an EKG monitor.

For the foregoing applications, it may be desirable to employee a series of electroporation electrodes along the length of a catheter shaft to affect a more linear region of tissue. For example, one may substitute the electrodes described in U.S. Pat. No. 6,161,543 to Cox et al, for electroporation element 125 of catheter 122, and substitute the energy generator of that patent for pulse generator 121 described above. Alternatively, the generator of the foregoing patent may be operated in a pulsed manner to achieve an electroporative effect. In the case of multiple linear electrodes, electrodes may be activated in pairs, in groups, or in a sequential manner in order to maximize the linearity of the lesion while minimizing the field strength requirements.

The apparatus and methods of the present invention also may be useful in treating all types of cardiac dysfunctions apart from atrial fibrillation. For example, the apparatus and methods present invention may be used to treat other electrophysiologic defects in the heart, or to create lesions for other purposes. One of the biggest advances in the treatment of congestive heart disease in recent years has been the introduction of implantable biventricular pacemakers. While there are many etiologies to congestive heart failure (CHF), it has been shown that dyssynchrony between the chambers of the heart is a significant cause of impaired ejection fractions. Biventricular pacemakers restore correct synchronizations between the chambers of the heart, improving pump efficiency and increasing ejection fraction and cardiac output.

Biventricular pacemakers, while a significant advance in the treatment of CHF, suffer from some significant drawbacks. For example, permanent implants carry with them a risk of infection. In addition, battery life is limited, and replacement of pacemakers requires additional surgical intervention. Further, placement of biventricular pacing leads requires more skill and is subject to more failure than placement of single-chamber leads. This is due not only to their increased number, but also to the specific locations in which the leads must be placed. For example, a pacing lead must be placed within the transverse coronary sinus, a location which is not simple to access, and in which there is limited experience with permanently implanted devices. Long-term effects of this implant may include occlusion of the sinus and erosion of the walls of the sinus.

One aspect of the present invention includes locating and isolating the nerves which control the beating of the dyssychronous chambers, and selectively ablating those nerves or a subset of those nerves, in order to alter the rhythm and/or rate of that chamber to bring it back into synchronicity with the other chambers. For example, it is known that a richly innervated fat pad (CS fat pad 18) runs along the path of the transverse coronary sinus. According to some embodiments of the present invention, an ablation catheter is inserted into the coronary sinus, the desired nerve bundles are located within the CS fat pad, and energy is directed to ablate the desired nerve bundles to change the rate and/or rhythm of the heart. Ablation of other fat pads and pathways will affect various dyssynchronies and are within the scope of the invention.

As another example, it is known that nerves important in the stimulation and blocking of ventricular tachycardia run along the right ventricular outflow tract. Identifying and ablating these nerves with an ablation catheter attenuates or eliminates ventricular tachycardia. Modification of these dysrhythmias alone or in connection with selective denervation and modification of other dysrhythmias tend to bring the chambers of the heart back into synchronicity and improve pump efficiency, ejection fraction and cardiac output.

According to the principles of the present invention, a wide variety of energy modes may be used to create lesions using epicardial, intravascular, esophageal or intracardiac probes. Radio-frequency electrical energy (monopolar and bipolar), microwaves, cryothermia probes/balloons, alcohol injection, laser light, magnetic and ultrasound energy are just a few of the technologies that may be used to stimulate, target and ablate fat pad tissues in the examples described in the present invention. In addition, other chemical agents, such as phenol, may be injected into selected areas to cause nerve block. The injection of chemical agents may require repetitive injections over time to be effective. These injections may be delivered using an implantable drug infusion pump, programmed to inject said chemical agent at pre-determined time intervals and doses in order to maintain the nerve block over extended periods of time.

In addition, energy such as PEFs to create electroporative effects at the cellular level of tissue or nerve structures also may be employed, as described above. In certain configurations it may be advantageous to use the electroporation catheter of the present invention in conjunction with a nerve blocking agent such as botox, capsaicin or other chemical or therapeutic agents. In this case, the voltage applied to the electrode elements would be in the range applicable to create a reversible electroporation of the nerve or tissue cells, thereby porating the cell to allow the therapeutic agent to be delivered to achieve the desired effect, but not destroy the cell or otherwise irreversibly damage the targeted tissue or nerve structures.

In other configurations, voltages may be applied via the electroporation catheter to induce irreversible electroporation without requiring the use of any other agents to achieve the desired cell destruction and/or denervation. It is a further advantage of this type of energy that any thermal effect may be minimized, thereby allowing the energy field to be sustained for a longer period of time than with the use of direct thermal energies, resulting in a larger or deeper treatment region depending on the electrode configuration utilized. Techniques of the present invention may destroy not only the fat pads, but also the targeted cardiac nerves. To aid the electroporation process, it may be advantageous to heat the targeted cells or surrounding tissue by either applying thermal energy directly to the region, or directing a heated fluid, such as saline to the region.

Although preferred illustrative embodiments of the present invention are described above, it will be evident to one skilled in the art that various changes and modifications may be made without departing from the scope of the invention. It will also be apparent that various changes and modifications may be made herein without departing from the invention. The appended claims are intended to cover all such changes and modifications that fall within the true spirit and scope of the invention.

1. Apparatus for selective denervation of heart tissue, comprising:
a first catheter having one or more electrodes disposed at a distal tip thereof;
a second catheter having one or more electrodes disposed at a distal tip thereof;
wherein the catheters are used to stimulate, target, and ablate fat pad tissue in order to selectively denervate heart tissue.

2. The apparatus of claim 1, wherein:
the first catheter is positioned within the right pulmonary artery;
the second catheter is positioned within the superior vena cava; and
the catheters are used to ablate the SVC-Ao fat pad.

3. The apparatus of claim 1, wherein:
the first catheter is positioned within the right atrium;
the second catheter is positioned within the right superior pulmonary vein; and
the catheters are used to ablate the RPV fat pad.

4. The apparatus of claim 1, wherein:
the first catheter is positioned within the right atrium;
the second catheter is positioned within the right inferior pulmonary vein; and
the catheters are used to ablate the RPV fat pad.

5. The apparatus of claim 1, wherein:
the first catheter is positioned within the inferior vena cava;
the second catheter is positioned within the coronary sinus; and
the catheters are used to ablate the IVC-ILA fat pad.

6. Apparatus for selective denervation of heart tissue, comprising:
a catheter having one or more electrodes disposed at a distal tip thereof;
wherein the catheter is used to stimulate, target, and ablate fat pad tissue in order to selectively denervate heart tissue.

7. The apparatus of claim 6, wherein:
the catheter is positioned within the esophagus; and
the catheter is used to ablate the LA, RPV, SVC-Ao, CS and/or IVC-ILA fat pad.

8. The apparatus of claim 6, wherein:
the catheter is positioned within the superior vena cava; and
the catheter is used to ablate the SVC-Ao fat pad.

9. The apparatus of claim 8, wherein the catheter is moved between the IVC-RA-SVC in order to ablate the SVC-Ao, RPV, IVC-ILA, CS fat pads

10. A method for selective denervation of heart tissue, comprising the steps of:
providing a first catheter having one or more electrodes disposed at a distal tip thereof;
providing a second catheter having one or more electrodes disposed at a distal tip thereof;
stimulating, targeting, and ablating fat pad tissue in order to selectively denervate heart tissue.

11. The method of claim 10, further comprising the steps of:
positioning the first catheter within the right pulmonary artery;
positioning the second catheter within the superior vena cava; and
ablating the SVC-Ao fat pad using both catheters.

12. The method of claim 10, further comprising the steps of:
positioning the first catheter within the right atrium;
positioning the second catheter within the right superior pulmonary vein; and
ablating the RPV fat pad using both catheters.

13. The method of claim 10, further comprising the steps of:
positioning the first ablation catheter within the right atrium;
positioning the second ablation catheter within the right inferior pulmonary vein; and
ablating the RPV fat pad using both catheters.

14. A method for selective denervation of heart tissue, comprising the steps of:
providing an ablation catheter having one or more electrodes disposed at a distal tip thereof;
stimulating, targeting, and ablating fat pad tissue in order to selectively denervate heart tissue.

15. The method of claim 14, further comprising the steps of:
positioning the ablation catheter within the esophagus; and
ablating the LA RPV, SVC-Ao, CS and/or IVC-ILA fat pad.

16. The method of claim 14, further comprising the steps of:
positioning the ablation catheter within the superior vena cava; and
ablating the SVC-Ao fat pad.

17. The method of claim 16, wherein the catheter is moved between the IVC-RA-SVC in order to ablate the SVC-Ao, RPV, IVC-ILA, CS fat pads.
